(19) United States
(12) Patent Application Publication
(10) Pub. No.: US 2024/0110195 A1
(43) Pub. Date: Apr. 4, 2024

(54) METHOD FOR TERRESTRIAL CARBON SEQUESTRATION

(71) Applicant: Colorado State University Research Foundation, Fort Collins, CO (US)

(72) Inventor: Richard Conant, Fort Collins, CO (US)

(21) Appl. No.: 18/486,766

(22) Filed: Oct. 13, 2023

Related U.S. Application Data
(63) Continuation of application No. PCT/US2022/024834, filed on Apr. 14, 2022.
(60) Provisional application No. 63/174,856, filed on Apr. 14, 2021.

(51) Int. Cl. C12N 15/82

(52) U.S. Cl. CPC C12N 15/8216

(57) ABSTRACT

Disclosed herein are methods of carbon sequestration using microorganisms, such as soil microorganisms, capable of synthesizing or consuming degradation-resistant L-carbohydrates. In particular, methods of identifying and using microorganisms capable of synthesizing L-carbohydrates are provided, wherein microorganisms that synthesize L-carbohydrates are contacted with a soil source to sequester carbon. Also provided are methods of identifying and incorporating biomolecules such as genes, peptides, and enzymes relevant for L-carbohydrate anabolism or catabolism into plants so the plants synthesize L-carbohydrates or do not consume L-carbohydrates, thereby sequestering carbon.

### CROSS-REFERENCE TO RELATED APPLICATIONS

This is a By-Pass Continuation Application claiming priority to PCT/US2022/024834, filed Apr. 14, 2022, which application claims priority under 35 U.S.C. § 119 to provisional application Ser. No. 63/174,856 filed Apr. 14, 2021.

### STATEMENT REGARDING FEDERALLY SPONSORED RESEARCH

This invention was made with government support under Grant No. 2020-7030-31475 awarded by the United States Department of Agriculture. The United States government has certain rights in this invention.

### TECHNICAL FIELD

Disclosed herein are novel methods of carbon sequestration utilizing plants or microorganisms capable of synthesizing degradation-resistant L-carbohydrates. These plant microorganisms have a variety of uses for carbon sequestration. More particularly, methods of collecting an environmental sample, such as a soil sample, with preferred microorganisms are provided, along with methods of identifying, classifying, and isolating said preferred microorganisms. Utilizing isolated soil microorganisms, methods of identifying and characterizing L-carbohydrate catabolic and anabolic pathways and substrates are disclosed, along with methods of genetic characterization to identify key genes involved in the regulation of L-carbohydrate anabolism and catabolism. These methods can be used to contact preferred microorganisms with a soil source or other target (e.g., a plant) to sequester carbon. They can also be used to convert carbohydrates or other biomolecules into microorganism cells and/or plants. Conversion into microorganism cells and/or plants permits carbon sequestration on a global scale.

### TECHNICAL BACKGROUND

Despite rapid growth in renewable energy generation and expanding efforts to reduce greenhouse gas emissions, global emissions grew by more than 3% in 2018. The likelihood of keeping climate change below 2° C. worldwide has continued to recede and the timeframe for doing so grows shorter. Carbon storage in terrestrial ecosystems could make a substantial contribution to offsetting emissions by facilitating increased carbon dioxide fixation by plants or by enabling fixed carbon to remain in the vegetation, soils, or forest products for longer periods of time. Usually, this is accomplished through changing agronomic, forestry, or other land-use practices.

Terrestrial carbon sequestration has advanced incrementally over the last twenty years, but real and substantial barriers have prevented the widespread uptake of terrestrial carbon sequestration projects. To receive credit for carbon offsets, existing protocols governing offset allowance trading require that any carbon stored is additional to what would have happened in the absence of efforts to offset emissions (additionally) and attempt to minimize the risk that an offset activity in one place prompts emission increases in another (leakage).

Current approaches to terrestrial carbon sequestration at scale require the adoption of new land management practices across large land areas and the engagement of a huge number of landholders. In many cases, monetary (e.g., biochar) and other costs (e.g., increased food costs for vulnerable people) are prohibitively high. Even for the lower-cost options, modest and variable rates of carbon sequestration led to substantial verification and transaction costs.

It is important that new low-cost, easy-to-verify, scalable, and sustainable approaches to terrestrial carbon sequestration be implemented to combat the challenge of ever-growing global emissions.

These and other objects, advantages, and features of the present disclosure will become apparent from the following specification taken in conjunction with the claims set forth herein.

### BRIEF SUMMARY

Disclosed herein are novel methods of carbon sequestration utilizing plants or microorganisms capable of synthesizing degradation-resistant L-carbohydrates. The soil microorganisms have a variety of uses for carbon sequestration. More particularly, methods of collecting an environmental sample (such as a soil sample or a sample of plant-associated microorganisms from a plant) with preferred microorganisms are provided, along with methods of identifying, classifying, and isolating said preferred microorganisms. Utilizing isolated soil microorganisms, methods of identifying and characterizing L-carbohydrate catabolic and anabolic pathways and substrates are disclosed, along with methods of genetic characterization to identify key genes involved in the regulation of L-carbohydrate anabolism and catabolism.

Disclosed herein are methods of sequestering carbon comprising: providing a composition comprising one or more plants or microorganisms incapable of decomposing at least one L-carbohydrate; and contacting the composition with a soil source.

In an embodiment, the at least one L-carbohydrate comprises an L-pentose, L-hexose, L-heptose, or any polymeric combination thereof. In a further embodiment, at least one L-carbohydrate comprises L-altrose, L-glucose, L-gulose, L-idose, L-mannose, L-ribulose, L-sorbose, L-talose, L-xylose, L-allose, L-fructose, L-galactose, L-psicose, or any polymeric combination thereof.

According to some embodiments, the composition further comprises one or more plants or microorganisms capable of synthesizing at least one L-carbohydrate or L-carbohydrate polymer.

In an embodiment, the one or more microorganisms comprise a bacterium from the phyla Actinobacteria, Proteobacteria, Acidobacteria, Firmicutes, Crenarchaeota, Chloroflexi, Myxococcota, Verrucomicrobia, Bacteroidetes, Gemmatimonadetes, Nitrospirota, Planctomycetes, Proteobacteria, or other or a combination thereof.

In some embodiments, the methods comprise or further comprise isolating the one or more microorganisms from a sample, preferably a soil sample. In an embodiment, the isolation of the one or more microorganisms comprises: collecting a sample from a soil source (e.g., a soil sample) or a plant source (e.g., one or more plant-associated microorganisms); serially diluting the sample to form a suspension comprising one or more microorganisms; growing the one or more microorganisms on a growth media; selecting one or more microorganisms incapable of decomposing at least one L-carbohydrate; and separating the one or more microorganisms from the growth media.

Also disclosed herein are methods of sequestering carbon in a transgenic plant comprising: a) introducing into a plant, plant part, or plant cell a DNA expression cassette comprising, a DNA sequence that encodes a protein capable of modifying the plant, plant part, or plant cell's utilization of a substrate in a metabolic pathway, wherein the substrate comprises a metabolite of an L-carbohydrate or a polymeric carbohydrate-containing L-carbohydrate monomer; and b) generating a plant cell that has at least one product of the metabolic pathway.

In some embodiments, the product of the metabolic pathway is an L-carbohydrate. In a further embodiment, the L-carbohydrate comprises an L-pentose, L-hexose, L-heptose, or a polymeric combination thereof. In a still further embodiment, the L-carbohydrate comprises L-altrose, L-glucose, L-gulose, L-idose, L-mannose, L-ribulose, L-sorbose, L-talose, L-xylose, L-allose, L-fructose, L-galactose, L-psicose, or a polymeric combination thereof.

According to some embodiments, the plant, plant part, or plant cell is all or a part of a tree, shrub, herb, grass, fern, moss, or agricultural crop. In a further embodiment, the agricultural crop is corn, rice, wheat, soybean, sorghum, wheat bran, or other commonly grown food, feed, or fodder crops.

Further disclosed herein are methods of converting growth media carbohydrates into an L-carbohydrate into one or more microorganisms comprising: introducing one or more growth substrates into an environment that comprises cells of the one or more microorganisms in a growth media; wherein one or more carbohydrates are used as a carbon source by the cells of the one or more microorganisms in the growth media for growth or biosynthesis, and converting a part or all of the source carbon into one or more L-carbohydrates in one or more microorganisms within the environment via a carbon-fixing reaction.

In an embodiment, the L-carbohydrate comprises an L-pentose, L-hexose, L-heptose, or a combination thereof. In a still further embodiment, the L-carbohydrate comprises L-altrose, L-glucose, L-gulose, L-idose, L-mannose, L-ribulose, L-sorbose, L-talose, L-xylose, L-allose, L-fructose, L-galactose, L-psicose, or a combination thereof.

In some embodiments, the one or more microorganisms comprise a bacterium, archaeon, fungus, alga, protozoan, virus, or a combination thereof. In a further embodiment, the bacterium comprises Actinobacteria, Proteobacteria, Acidobacteria, Firmicutes, Crenarchaeota, Chloroflexi, Myxococcota, Verrucomicrobia, Bacteroidetes, Gemmatimonadetes, Nitrospirota, Planctomycetes, Proteobacteria, or a combination thereof.

Further disclosed herein are kits for detecting and identifying one or more microorganisms capable of synthesizing an L-carbohydrate and/or not decomposing an L-carbohydrate comprising: a) a composition comprising at least one primer pair comprising a forward primer and a reverse primer, suitable for amplifying a locus involved in the synthesis of an L-carbohydrate and/or the inability to digest an L-carbohydrate; b) a composition comprising at least one nucleic acid probe capable of hybridizing to the locus; c) a buffer suitable for hybridization a reaction between a nucleic acid target and the probe and/or primer pair, wherein the reaction forms one or more hybridized nucleic acids; d) a solution for washing the one or more hybridized nucleic acids; and e) optionally, a means for detection of the one or more hybridized nucleic acids.

In an embodiment, the L-carbohydrate comprises an L-pentose, L-hexose, L-heptose, or a combination thereof. In a still further embodiment, the L-carbohydrate comprises L-altrose, L-glucose, L-gulose, L-idose, L-mannose, L-ribulose, L-sorbose, L-talose, L-xylose, L-allose, L-fructose, L-galactose, L-psicose, or a combination thereof.

In some embodiments, the one or more microorganisms comprise a bacterium, archaeon, fungus, alga, protozoan, virus, or a combination thereof. In a further embodiment, the bacterium comprises Actinobacteria, Proteobacteria, Acidobacteria, Firmicutes, Crenarchaeota, Chloroflexi, Myxococcota, Verrucomicrobia, Bacteroidetes, Gemmatimonadetes, Nitrospirota, Planctomycetes, Proteobacteria, or a combination thereof.

Also disclosed are methods for detecting and/or identifying one or more microorganisms capable of synthesizing an L-carbohydrate and/or not decomposing an L-carbohydrate comprising: a) isolating a target DNA sequence involved in the synthesis of an L-carbohydrate and/or the inability to consume an L-carbohydrate; b) amplifying the target DNA sequence with at least one primer pair comprising a forward primer and a reverse primer, suitable for amplifying a locus involved in the synthesis of an L-carbohydrate and/or the inability to digest an L-carbohydrate;

c) contacting the target DNA sequence or a fragment thereof with a probe and/or a primer that hybridizes with the target DNA sequence to form a hybrid; d) detecting the hybrid, and e) identifying one or more microorganisms capable of synthesizing an L-carbohydrate and/or not decomposing an L-carbohydrate.

In an embodiment, the L-carbohydrate comprises an L-pentose, L-hexose, L-heptose, or a combination thereof. In a still further embodiment, the L-carbohydrate comprises L-altrose, L-glucose, L-gulose, L-idose, L-mannose, L-ribulose, L-sorbose, L-talose, L-xylose, L-allose, L-fructose, L-galactose, L-psicose, or a combination thereof.

In some embodiments, the one or more microorganisms comprise a bacterium, archaeon, fungus, alga, protozoan, virus, or a combination thereof. In a further embodiment, the bacterium comprises Actinobacteria, Proteobacteria, Acidobacteria, Firmicutes, Crenarchaeota, Chloroflexi, Myxococcota, Verrucomicrobia, Bacteroidetes, Gemmatimonadetes, Nitrospirota, Planctomycetes, Proteobacteria, or a combination thereof.

Further disclosed are microorganisms produced by a method of converting an L-carbohydrate into one or more microorganisms comprising: introducing one or more L-carbohydrates into an environment that comprises cells of the one or more microorganisms in a growth media; wherein the one or more L-carbohydrates are synthesized by the cells of the one or more microorganisms in the growth media for growth or biosynthesis and converting a part or all of the growth media energy source into one or more L-carbohydrates in the one or more microorganisms within the environment via a carbon-fixing reaction.

Also disclosed is a plant, plant part, or plant cell produced by a method of sequestering carbon in a transgenic plant comprising: a) introducing into a plant, plant part, or plant cell a DNA expression cassette comprising, a DNA sequence that encodes a protein capable of modifying the plant, plant part, or plant cell's utilization of a substrate in a metabolic pathway, wherein the substrate is used to synthesize an L-carbohydrate, and b) generating a plant cell that has at least one product of the metabolic pathway.

While multiple embodiments are disclosed, still other embodiments of the present disclosure will become apparent based on the detailed description, which shows and describes illustrative embodiments of the disclosure. The foregoing summary is illustrative only and is not intended to be in any way limiting. In addition to the illustrative aspects, embodiments, and features described above, further aspects, embodiments, and features of the present technology are apparent from the following drawings and the detailed description, which shows and describes illustrative embodiments of the present technology.

Each feature of the technology described herein may be combined with any one or more other features of the disclosure, e.g., the methods may be used with any hybrid material described herein. Accordingly, the drawings and detailed description are to be regarded as illustrative and not restrictive.

### BRIEF DESCRIPTION OF THE DRAWINGS

FIG. 1 depicts a table of selected L-carbohydrates.

FIG. 2 is a graph of cumulative CO₂—C respiration of ten L-carbohydrates.

FIG. 3 is a graph of cumulative CO₂—C respiration of four L-carbohydrates.

FIG. 4 is a chart comparing dominant bacteria taxa across various soil samples.

FIG. 5 is a pie chart showing the abundance-weighted percentage of shared phyla across various soil samples.

FIG. 6 is a map illustrating locations across the United States where soil samples were collected.

Various embodiments of the present disclosure will be described in detail regarding the drawings. Reference to various embodiments does not limit the scope of the disclosure. Figures represented herein are not limitations to the various embodiments according to the disclosure and are presented for exemplary illustration of the disclosure.

### DETAILED DESCRIPTION OF THE PREFERRED EMBODIMENTS

The present disclosure relates to biologically inactive carbohydrate enantiomers resistant to decomposition in the environment, and further capable of carbon sequestration including identification, isolation, and classification of microbes capable of both L-carbohydrate catabolism (CO₂ production) and L-carbohydrate synthesis. Classification of responsible organism/consortia using RNA extraction, sequencing, and other taxonomical methods may indicate responsible genes for L-carbohydrate synthesis and catabolism. Further, manipulation and/or combination of the indicated genes may be optimized to generate biologically inactive carbohydrate enantiomers resistant to decomposition and carbon sequestration.

The embodiments of this disclosure are not limited to particular types of materials or methods, which can vary. It is further to be understood that all terminology used herein is to describe particular embodiments only and is not intended to be limiting in any manner or scope. For example, as used in this specification and the appended claims, the singular forms “a,” “an” and “the” can include plural referents unless the context indicates otherwise.

Unless indicated otherwise, “or” can mean any one alone or any combination thereof, e.g., “A, B, or C” means the same as any of A alone, B alone, C alone, “A and B,” “A and C,” “B and C” or “A, B, and C.”

Further, all units, prefixes, and symbols may be denoted in their SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, a description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6, and decimals and fractions, for example, 1.2, 3.8, 1½, and 4¾ This applies regardless of the breadth of the range.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

The term “about,” as used herein, refers to variation in the numerical quantity that can occur, for example, through typical measuring techniques and equipment, with respect to any quantifiable variable, including, but not limited to, mass, volume, time, temperature, pH, reflectance, whiteness, etc. Further, given solid and liquid handling procedures used in the real world, there is certain inadvertent error and variation that is likely through differences in the manufacture, source, or purity of the ingredients used to make the hybrid materials or carry out the methods and the like. The term “about” also encompasses amounts that differ due to different equilibrium conditions for a hybrid material resulting from a particular initial mixture. The term “about” also encompasses these variations. Whether or not modified by the term “about,” the claims include equivalents to the quantities.

As used herein, an “isolated nucleic acid” is one that is separated from other nucleic acid molecules which are present in the natural source of the nucleic acid. For example, with regards to genomic DNA, the term isolated includes nucleic acid molecules which are separated from the chromosome with which the genomic DNA is naturally associated.

The term “probe” or “nucleic acid probe” refers to single-stranded sequence-specific oligonucleotides which have a base sequence that is sufficiently complementary to hybridize to a target base sequence to be detected.

As used herein, the term “primer” refers to a single-stranded DNA oligonucleotide sequence capable of acting as a point of initiation for the synthesis of a primer, an extension product that is complementary to the nucleic acid strand to be copied. The length and the sequence of the primer must be such that they allow to prime the synthesis of the extension products. Specific length and sequence will depend on the complexity of the required DNA or RNA targets, as well as on the conditions of primer use.

The term “target” or “target sequence” as used herein refers to nucleic acids originating from a biological sample to be identified and which preferably have a base sequence at least partially complementary to at least one nucleic acid probe. The target nucleic acid can be single- or double-stranded DNA.

As used herein, “sample” “environmental sample” and “biological sample” refer to a specimen such as an environmental sample comprising soil, a plant source such as a plant, plant part, plant cell, plant genetic material, or microorganisms associated with a plant, one or more microbes or colonies (e.g., microorganisms from soil), contaminated or pure cultures, or purified nucleic acids, preferably in which the target sequence of interest may be found. Use of one of the terms “sample,” “environmental sample” or “biological sample” in one instance does not exclude the scope of the other terms. That is, any discussion of “sample” should be understood to be inclusive of “environmental sample” and “biological sample” and vice versa.

As used herein, the term “plant” includes a whole plant and any descendant, cell, tissue, or part of a plant. The term “plant parts” includes any part(s) of a plant, including, for example, and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, flowers, or stalks. In contrast, some plant cells are not capable of being regenerated to produce plants and are referred to herein as “non-regenerable” plant cells.

The term “microorganisms” includes soil microorganisms and plant-associated microorganisms such as archaea, bacteria, fungi, and protozoa. Soil microorganisms are responsible for many enzymatic processes in soil and store energy and nutrients in their biomass. Plant-associated microbes, which include viruses, bacteria, fungi, oomycetes, and nematodes, contribute substantially to plant health. For example, plant-associated bacteria include, without limitation, endophytic, rhizospheric, and phyllospheric bacteria. Further examples of soil microorganisms and plant-associated microorganisms are described in Wasai and Minamisawa, Plant-Associated Microbes: From Rhizobia to Plant Microbiomes, Microbes Environ. 33(1), 2018; and Visser and Parkinson, Soil biological criteria as indicators of soil quality: Soil microorganisms, Am. J. of Alt. Ag. 7(1-2), 1997, both of which are herein incorporated by reference in their entirety. Reference to “soil microorganisms” as used herein does not exclude “plant-associated microorganisms” and vice versa. That is, any discussion of “soil microorganisms” should be understood to be inclusive of “plant-associated microorganisms” and vice versa.

As used herein, the term “polynucleotide” is not intended to limit the present disclosure to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogs including, but not limited to, nucleotide analogs or modified backbone residues or linkages, which are synthetic, naturally occurring, and non-naturally occurring, which have similar binding properties as the reference nucleic acid, and which are metabolized like the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The term polynucleotides also encompass all forms of polynucleotides including, but not limited to, single-stranded forms, double-stranded forms, hairpins, stem-and-loop structures, complements, and the like.

As used herein, the term “stable transformation” is intended that a polynucleotide introduced into a plant integrates into the genome of the plant and is capable of being inherited by progeny thereof. In contrast, “transient transformation” indicates that a polynucleotide introduced into a plant does not integrate into the genome of the plant.

As used herein, the term “transgenic plant” refers to a “plant” as described above, wherein the plant comprises a polynucleotide that is introduced into a plant (i.e., a transgene) by, for example, any of the stable and transient transformation methods disclosed elsewhere herein or otherwise known in the art. Such transgenic plants and transformed plants also refer, for example, the plant into which the polynucleotide was first introduced and also any of its progeny plants that comprise the polynucleotide.

### Carbon Sequestration

Carbon dioxide is the most commonly produced greenhouse gas. Carbon sequestration is the process of capturing carbon dioxide and storing that carbon from a part of the carbon cycle. The carbon cycle involves the reusing of carbon atoms, whereby carbon enters the atmosphere as carbon dioxide, the carbon dioxide is absorbed by autotrophs, said autotrophs are consumed by animals, and when the animals die their bodies decompose and carbon is reabsorbed back into the atmosphere. Generally, carbon is stored in major sinks as organic molecules in living or dead organisms in the biosphere, as carbon dioxide gas in the atmosphere, as organic matter in soils, in the lithosphere as fossil fuels and sedimentary rock deposits, and the oceans as dissolved carbon dioxide.

Microbes play a role in the carbon cycle. They transform the state of carbon by sequestering carbon from and releasing carbon into, the atmosphere, oceans, and biomass. Through carbon fixation, a byproduct of photosynthesis, microorganisms (e.g., soil microbes and marine microbes) incorporate carbon into their biochemical structures, thereby releasing oxygen as a byproduct and introducing carbon into the food chain.

Human activities have increased the abundance of carbon dioxide in the atmosphere by greater than 40% since the beginning of the industrial age. Once added to the atmosphere, carbon dioxide remains for hundreds of years. The increased carbon dioxide levels in the atmosphere also have a secondary effect of decreasing microbes' carbon sequestration abilities and in many cases increasing respiration. In particular, soil respiration is a measure of the carbon dioxide released from the soil as a result of the decomposition of soil organic matter by soil microbes and other fauna.

Terrestrial carbon sequestering provides a solution to address this problem. Generally speaking, terrestrial carbon sequestration is the process through which carbon dioxide from the atmosphere is absorbed by vegetation, such as trees and plants, through photosynthesis and stored as carbon in soils and biomass (for example, tree trunks, branches, foliage, roots, and the like). Soil carbon sequestration in particular refers to the uptake of carbon-containing substances from the atmosphere and their storage in soil carbon pools. The soil microbial community plays an important role in carbon cycling: microbial activity and health are a major force in the extent to which carbon can be stored in the soil.

Enhancing soil organic matter in soils has the potential to substantially offset carbon dioxide emissions by facilitating increased carbon fixation by plants or by enabling fixed carbon to remain in the vegetation or soil for longer periods of time, while also promoting soil health and resilience. The extent and rate of soil carbon release depend largely on soil microorganisms, which are a large driver of soil quality. As organic material is deposited on the soil, soil microorganisms begin the decomposition process.

The decomposition capabilities of soil microorganisms can therefore impact carbon sequestration through the anabolism and catabolism of carbon in the form of carbohydrates. As used herein, the term “carbohydrate” refers to a compound according to the general formula Cₓ(H₂O)_{y}, wherein x and y are variable integers. The name “carbohydrate” stems from the fact that hydrogen and oxygen are present in the same amounts as in water. Carbohydrates are divided into three primary classes: monosaccharides, disaccharides, and polysaccharides. Monosaccharides are single sugar units having a general formula of (CH₂O)ₙ wherein n is an integer between 3-9, and which are classified according to the number of carbons as trioses (3C), tetroses (4C), pentoses (5C), hexoses (6C), and heptoses (7C). Disaccharides have two units of monosaccharides connected by a glycosidic bond. Similarly, a trisaccharide has three monosaccharide units.

Decomposition experiments conducted by Louis Pasteur over 150 years ago revealed that biochemicals with a chiral center are mirror images of each other, but only one of them is biologically active. When compounds containing equal parts of both stereoisomers decompose, the mixture is transformed over time until it contains just one stereoisomer and decomposition subsequently stops. Carbohydrates, therefore, have both biologically active and biologically inactive configurations (or at least less biologically active). As sugars, the stereochemistry of sugars of often described using D and L notation, based on the chiral carbon farthest from the C═O carbonyl group (i.e., the penultimate carbon). In D-carbohydrates, the OH group on the chiral center furthest from the carbonyl is on the right. Comparatively, in L-carbohydrates, the OH group on the chiral center furthest from the carbonyl is on the left. Further discussion of homochirality can be found in Axel Brandenburg's Homochirality: A Prerequisite or Consequence of Life?, which is herein incorporated by reference in its entirety.

D-carbohydrates and L-carbohydrates can occur naturally, or they can be synthesized. Further discussion of methods of synthesizing L-sugars can be found in Fabien P. Boulineau and Alexander Wei, Synthesis of L-Sugars from 4-Deoxypentenosides, Org. Lett. 2002, 4, 13, 2281-2283, Jun. 5, 2002; Tian-Yu Xia et al, Synthesis of 1-glucose and 1-galactose derivatives from d-sugars, Chinese Chem. Letters, 25(9), 1220-1224, 2014; and Takahashi H., et al., Divergent synthesis of L-sugars and L-iminosugars from D-sugars, Chem. 12(22), Jul. 24, 2006, all of which are herein incorporated by reference in their entirety. When discussing carbohydrates herein, the L-carbohydrates may include L-carbohydrate monomers and/or polymers containing or associated with an L-carbohydrate.

In some embodiments, detection of various soil microbe's ability to consume L-carbohydrates may include the use of a single L-carbohydrate carbon source. For example, L-carbohydrates useful for carbon sequestration are five-, six-, or seven-carbon sugars which are chiral and water-soluble. Suitable pentose L-carbohydrates include, without limitation ribose and ribulose. Suitable hexose L-carbohydrates include, without limitation, allose, glucose, mannose, talose, fructose, and psicose. Suitable heptose L-carbohydrates include, without limitation sedoheptulose and mannoheptulose. In particular, allose and talose are sugars that occur in both L- and D-conformations.

Soil microbes may have evolved the ability to synthesize L-sugars, though this ability appears to be very rare. Similarly, the ability of soil microbes to consume L-sugars also appears rare. L-Carbohydrates are slow to be decomposed compared to their corresponding D-enantiomers. Some L-carbohydrates are found and synthesized naturally, although it is rare and challenging to find organisms that anabolize and/or catabolize L-carbohydrates. Interestingly, some soil bacteria and other microorganisms demonstrate the ability to catabolize L-carbohydrates while other soil microorganisms do not. This differential capability of soil microorganisms to catabolize L-carbohydrates suggests underlying genetic factors control L-carbohydrate synthesis and catabolism.

Identification of genes and corresponding proteins responsible for the inability to catabolize L-carbohydrates presents an important opportunity for carbon sequestration. Soil microorganisms not capable of catabolizing L-carbohydrates can be added to any suitable soil source to improve the quality of the soil and make carbon less available for plants and other organisms. Further, the genes responsible for the poor catabolism of L-carbohydrates can be selectively added to plants via gene editing techniques and/or breeding to increase L-carbohydrate synthesis in various plants. The failure to, or the slow process of catabolizing L-carbohydrates means that the carbon in the L-carbohydrates remains sequestered in the soil, rather than released into the atmosphere by plant respiration.

Further, the soil microorganisms capable of catabolizing L-carbohydrates are likely to have the capability of synthesizing L-carbohydrates. Soil microorganisms capable of synthesizing, but not catabolizing, L-carbohydrates can be added to a soil source to improve the quality of the soil and ultimately make much less carbon available as these microorganisms are synthesizing L-carbohydrates which will not be easily broken down and released as carbon. Further, identification of the genes responsible for L-carbohydrate anabolism also presents an important opportunity for carbon sequestration. The genes responsible for the synthesis of L-carbohydrates can be selectively added to plants via gene editing techniques and/or breeding to increase the production of L-carbohydrates.

Thus, it should be possible to devise a system to efficiently produce decomposition-resistant L-sugars. The methods of using microorganisms and/or agricultural crops to sequester carbon via L-carbohydrates could lead to massive biological carbon sequestration. Free solar energy can effectively be used to fix atmospheric carbon dioxide and pump stable, natural carbon compounds into agricultural soils and/or products at no cost to farmers or land caretakers. Plants exude as much as 20% of the carbon they fixed by photosynthesis in the soil. If just a fraction of these labile exudates (say 10%) are replaced with decomposition-resistant L-sugars, the resulting carbon storage rates would be tremendous—approximately 0.5 tC/ha/yr, effectively about that which could be achieved with the adoption of no-tillage, cover crops, fallow elimination, and other high carbon-sequestering practices. The carbon storage potential of croplands alone is enormous. Importantly, the methods described herein can be employed anywhere that plants are sown.

In sum, novel methods of soil carbon sequestration are described herein, whereby soil microorganisms capable of synthesizing and/or consuming decomposition-resistant L-carbohydrates are isolated. These microorganisms fix carbon in the form of L-carbohydrates which are decomposition resistant and/or the microorganisms simply fail to decompose L-carbohydrates, thereby slowing the rate of carbon release as decomposition occurs. These microorganisms may be added to a source of soil to slow carbon dioxide release and promote soil health. Additionally, genes and proteins responsible for L-carbohydrate consumption and synthesis can be transferred into plants, thereby magnifying the scope and effects of terrestrial carbon sequestration.

### Methods of Environmental Sample Collection

The disclosure provides methods of collecting environmental samples, such as soil samples, and particularly samples having one or more microorganisms capable of synthesizing or consuming L-carbohydrates. There are abundant microorganisms thriving in soil. It is well known that a considerable number of bacterial and fungal species possess a functional relationship with animals and particularly plants. Soil microorganisms are important components in the natural soil sub ecosystem because not only can they contribute to nutrient availability in the soil, but they also help bind soil particles into stable aggregates, which improve soil structure and reduce erosion potential.

Although soil bacteria have been studied for many decades, the nature and diversity of most of these bacteria remain unknown. Despite the extent to which soil bacteria contribute to major ecosystem processes such as nutrient and carbon cycling, plant production, and greenhouse gas emissions, further characterization of these bacteria remains a challenge. First, soil bacteria are among the most abundant and diverse groups of organisms on earth, making characterizing difficult due to sheer volume. Additionally, many soil bacteria do not match those found in preexisting 16S rRNA gene databases and are challenging to cultivate in vitro, making identification, classification, and sequencing difficult. For these reasons, the primary attributes, environmental preferences, and metabolic capabilities of most soil microorganisms remain unknown.

Soil collection can occur from a broad range of environments. The soil samples included may span a wide range of vegetation types, bioclimatic regions, and edaphic characteristics. The diversity of samples may increase the potential number of soil microbial taxa identified, to further allow the identification of millions of soil microbial taxa that consume and/or produce L-carbohydrates. Soil collection may be collected from Long-Term Ecological Research (LTER) sites, Long-Term Agricultural Research (LTAR) sites, National Environmental Observatory Network (NEON) sites, National Forests, Grasslands, and Park sites, or any other environments. The resulting collection may represent a wide range of climate, soil characteristics, and vegetation types, including croplands, wetlands, or any other environment. Soil collected may be characterized and archived as a storehouse of soil biological diversity.

Overall, environmental sample collection occurs to identify and characterize microorganisms capable of synthesizing and/or consuming L-carbohydrates. In a preferred embodiment, the soil samples collected having microorganisms capable of synthesizing and/or consuming L-carbohydrates are from a variety of different climate and soil characteristics, as it is preferable to utilize microorganisms already suited to targeted regions for carbon sequestration.

In a preferred embodiment, samples are collected from the United States of America, more preferably Alaska, Washington, Oregon, California, Nevada, Utah, Idaho, Colorado, Oklahoma, Nebraska, Minnesota, Missouri, Michigan, Pennsylvania, Connecticut, Delaware, West Virginia, and/or Virginia.

### Preparation of Isolates Comprising Microorganisms Capable of Synthesizing or Consuming L-Carbohydrates

Methods for the isolation of microorganisms having the ability to consume L-carbohydrates or to synthesize L-carbohydrates are provided. Individual isolates are contemplated for further testing and use individually and in combinations. In one embodiment, the microbial isolate is combined with other isolates provided herein for providing formulations of the present inventions. In another embodiment, the microbial isolate is combined in sub formulations of the present inventions. In even further embodiments, the microbial isolate is combined in any combination with other isolates in order to provide formulations of the present inventions. In particular, individual microorganism isolates may be provided by serial diluting mixtures and plating for isolation of single colonies followed by growing isolates for identification (e.g., rDNA identification).

In an embodiment, a method of isolating a microorganism is provided comprising collecting a sample from a soil source or a plant source, serially diluting the sample to form a soil suspension comprising one or more microorganisms, growing the one or more microorganisms on a growth media, identifying the one or more microorganisms, selecting one or more preferred microorganisms, and separating the one or more preferred microorganisms from the growth media. In an embodiment, the one or more preferred microorganisms comprise microorganisms capable of synthesizing or consuming L-carbohydrates.

In other embodiments, detection of various soil microbes' abilities to synthesize L-carbohydrates may include the use of general growth media to encourage the growth of a variety of soil microbes, not only those that consume L-carbohydrates.

Soil Microbe Identification and Classification by 16S rRNA Sequencing

Soil microbial taxa identified may belong to any number of microorganism populations found in soil, i.e., soil algae, archaea, bacteria, actinomycetes, bacteriophages, protozoa, nematodes, fungi, or any other microorganisms. In some embodiments, a high-throughput screening approach may be used to assay the functional attributes of soil microbes. Microplate-reader and functional assays, such as Tecan Infinite M200 and Microresp, respectively, among other technology and techniques may be used, as otherwise known in the art. Examples of methods of extracting soil microbe genome DNA and total RNA are found in CN 101974513, which is herein incorporated by reference in its entirety.

In general, methods of soil microbe or plant-associated microbe identification and classification may occur by first identifying the most dominant bacterial phylotypes by 16S rRNA gene amplicon sequencing. Bacterial phylotypes may be sorted by Dominant phylotypes (taxa that share ≥97% sequence similarity across the amplified 16S rRNA gene region) include those that are highly abundant (top 10% most common phylotypes sorted by their percentage of 16S rRNA reads) and found in a large portion of environmental samples evaluated (e.g., more than half). When characterizing many samples, particularly global samples, it is likely that only a small fraction of phylotypes will be shared across samples, particularly for soil samples, and most phylotypes were relatively rare. Phylotype identification can optionally be cross-validated by using other approaches (e.g., shotgun metagenomic approach) and/or comparing the results of a collected sample set with another known database, such as those in the Earth Microbiome Project (EMP).

Preferably, the soil microorganisms are one or more microorganisms in the phyla Actinobacteria, Proteobacteria, Acidobacteria, Firmicutes, Crenarchaeota, Chloroflexi, Myxococcota, Verrucomicrobia, Bacteroidetes, Gemmatimonadetes, Nitrospirota, Planctomycetes, or Proteobacteria. Thus, in accordance with the disclosure, a number of genetic regions/loci may be identified and characterized, enabling the identification and genotyping of microorganisms in the phyla Actinobacteria, Proteobacteria, Acidobacteria, Firmicutes, Crenarchaeota, Chloroflexi, Myxococcota, Verrucomicrobia, Bacteroidetes, Gemmatimonadetes, Nitrospirota, Planctomycetes, or Proteobacteria.

### Methods of Genetic Characterization to Identify Target Loci, Primers, and Probes

Once relevant microorganisms and enzymatic pathways are identified, manipulation and/or combination of the relevant genes may be conducted to generate biologically inactive carbohydrate enantiomers resistant to decomposition and carbon sequestration. In some embodiments, soil DNA extracting, using techniques such as that of the MO-BIO PowerSoil DNA isolation kit, or other similar techniques known in the art, may be performed to amplify genes via polymerase chain reaction (PCR) using a distinct barcoded primer for each sample. In further embodiments, sequencing may be performed using Illumina MiSeq technology, or other similar techniques known in the art, and analyzed using the Quantitative Insights Into Microbial Ecology (QIIME) pipeline to identify operational taxonomic units, taxonomical assignment, and other analyses including assessments. Suitable methods, particularly for soil microbes, are described in Shimizu et al.

In some embodiments, DNA extraction and sequencing, and genome comparison are used for microorganism typing loci. Such methods involve first collecting sample microorganisms and optionally one or more reference microorganisms. The genomes of the sample microorganisms and optionally reference microorganisms can be used in a multi-genome comparison study to search for informative typing loci. If available and/or suitable, each genomic sequence may be compared to a reference genome, for example, reference genomes in publicly available databases such as GenBank, RefSoil/RefSeq, the NCBI 16S microbial blast database, and/or the Genomes Online Database (GOLD). This comparison can be conducted using any suitable tool (e.g., software) for comparing primary biological sequence information, particularly for the alignment of 16S amplicon sequences, such as BLASTN, BLASTx, Lambda, SINA/SILVA, and the like. Then, common homologous regions (CHRs) among the genomes are extracted. The parameter for CHRs will vary depending on the particular microorganisms evaluated, and further information on sequence similarity (“homology”) search is provided in Pears, An Introduction to Sequence Similarity (“Homology”) Searching, Curr. Protoc. Bioinformatics, 2013, which is herein incorporated by reference in its entirety. The coordinates of the CHRs are then marked. Amplification primers for typic loci are then determined from the multiple sequence alignments of CHRs.

Following the multiple genome comparison, DNA from sample microorganisms may be extracted and amplified via any suitable method (e.g., PCR with Taq DNA polymerase) using the amplification primers. PCR products are then purified and sequenced from both ends with the same amplification primers using any suitable platform, for example, short read Illumina (e.g., Illumina HiSeq, MiSeq) or any comparable next-generation sequencing (NGS) technology, wherein sequences are produced by attaching adapters to the end of short DNA fragments followed by a bridge amplification step and finally, the sequences are determined by sequencing by synthesis, one nucleotide at a time, with fluorescently tagged dNTPs. Alternatively, PacBio long-ready technology, also referred to as Single Molecule Real-Time (SMRT) sequencing or comparable technology may be used, which involves monitoring the activity of DNA polymerase molecules attached to the base of zero-mode-waveguides (ZMWs) using fluorescent-labeled nucleotides. Other sequencing platforms include, without limitation, Ion torrent/proton systems (e.g., those from Thermo Fisher) and Oxford Nanopore MinIon/GridION/Flongle long read sequencing technology, and the like.

Nucleotide sequences may be assembled using any suitable software and technique. Assembly software creates a representation of the actual genome from the raw sequencing read data which represent fragmented pieces of the genome with each genomic region on average covered multiple times. The resulting genome assembly consists of a variable number of continuous sequences referred to as “contigs” that together represent most of the genome. Some parts of the genome usually remain unresolved in the form of gaps between the contigs. Techniques such as paired-end sequencing or mate-pair sequencing are non-limiting examples of suitable techniques and can generate information that can link contig ends via a stretch of unknown sequence, a spanned gap. An assembly containing spanned gaps between at least some of the contigs is typically submitted as “scaffolds.” Other suitable genome projects resolve all gaps and uncertainties resulting in a “complete genome.” Examples of assembly software include, without limitation, phredPhrap, SPAdes, SKESA, or software suites developed by commercial entities (e.g., CLC bio, EvoCAT, etc.). Further discussion and comparison of sequencing and assembly platforms and methods are provided in Sergman, The Most Frequently Used Sequencing Technologies and Assembly Methods in Different time Segments of the Bacterial Surveillance and RefSeq Genome Databases, Front. Cell. Infect. Microbiol., 2020, which is herein incorporated by reference in its entirety.

The assembled genomes may then undergo alignment using any suitable software, for example, Sequencher, BioEdit, SATe, ProbCons, MAFFT (L0INSi), Clustal Omega, T-Coffee, and/or any of the platforms described herein (e.g., BLAST). Phylogenetic analysis may then be conducted to develop a phylogenetic tree of each target locus and congruencies among trees. This analysis may be done using, for example, the Quantitative Insights Into Microbial Ecology (QIIME) pipeline to identify operational taxonomic units and taxonomical assignment and/or Conscnsc from the phylip-3.69 package.

From this systematic comparison and analysis of microorganism genomes, genomic regions/target loci may be identified which assist in typing the microorganisms and determining evolutionary development of the same. More particularly, target CHRs are identified from the results of the phylogenetic analysis. Optionally, two or more adjacent CHRs may be combined into one locus. Preferably, target loci include, are adjacent to, or otherwise relate to protein-coding genes that impact carbohydrate metabolic pathways, preferably L-carbohydrate anabolism or catabolism.

### Primers and Probes

Further, primers and probes can be derived from the nucleic acid sequences comprising the target loci. Thus, the disclosure provides primer pairs (forward and reverse primers) suitable for amplifying a locus (e.g., the sequence of any one of the target loci). The primers may be any suitable length. For example, in some embodiments, the primers are at least 9 nucleotides in length and can be as long as about 50 nucleotides. In further embodiments, the primer may be, for example, at least 15 nucleotides in length and has at least 70%, 80%, 90%, or more than 95% identity to the full complement of the target sequence. Of course, primers consisting of more than 50 nucleotides can be used.

The methods of sequencing and characterizing target loci also relate to a nucleic acid probe capable of hybridizing to a locus described herein (e.g., the sequence of any one of the target loci). The probes may be any suitable length. For example, in some embodiments, the probes are at least 9 nucleotides in length and have at least 70%, 80%, 90%, or more than 95% identity to the complement of the target sequence to be detected. In some embodiments, probes are about 15 to 50 nucleotides long. As also disclosed herein, the primers and probes can be used for diagnostic purposes, in investigating the presence or the absence of a target nucleic acid in a biological sample, according to all the known hybridization techniques such as, for example, dot blot, slot blot, hybridization on arrays, etc. The probes will preferably hybridize specifically to one or more of the above-mentioned loci. An extensive guide to the hybridization of nucleic acids is found in Tijssen (1993) Laboratory Techniques in Biochemistry and Molecular Biology Hybridization with Nucleic Acid Probes, Part 1, Chapter 2 (Elsevier, New York); Ausubel et al., eds. (1995) Current Protocols in Molecular Biology, Chapter 2 (Greene Publishing and Wiley-Interscience, New York); and Sambrook et al. (1989) Molecular Cloning: A Laboratory Manual (2d ed., Cold Spring Harbor Laboratory Press, Plainview, N.Y.), all of which are incorporated by reference in their entirety.

### Compositions and Kits

The nucleic acid probes described herein can be included in a composition or kit which can be used to rapidly determine the presence or absence of a specific locus. In some embodiments, the kit comprises a composition comprising at least one primer pair (forward and reverse primers) suitable for amplifying a locus. In yet another embodiment, the composition comprises at least one nucleic acid probe capable of hybridizing to a target locus identified by the methods disclosed herein. By composition, it is meant that primers or probes complementary to the loci described herein may be in a pure state or in combination with other primers or probes. In addition, the primers or probes may be in combination with salts, pH buffers, stabilizing agents, DNA protection buffers, and may be in a dried state, in an alcohol solution as a precipitate, or an aqueous solution. Methods for collecting and media for storing nucleotide sequences can be found, for example, in U.S. Pat. No. 9,416,416, which is herein incorporated by reference in its entirety.

In a still further embodiment, a kit for detecting and identifying one or more microorganisms in a sample comprises a) a composition comprising at least one primer pair (forward and reverse primers) suitable for amplifying a locus described herein; b) a composition comprising at least one nucleic acid probe capable of hybridizing to a locus described herein; c) a buffer suitable for hybridization reactions between the probes or primers and nucleic acid targets in a sample; d) a solution for washing hybridized nucleic acids formed under the appropriate wash conditions or components necessary for producing the solution, and e) optionally a means for detection of said hybrids.

### Assays

As described herein, the methods of sequencing and characterizing target loci also relate to assays for detecting and identifying one or more microorganisms in a sample, wherein the assay comprises the use of at least one of the target loci/genetic regions identified by the methods described herein. In an embodiment, embodiment, the method for detecting and identifying one or more microorganisms comprises the following steps: a) optionally isolating and/or concentrating the DNA present in a sample; b) amplifying the DNA with at least one pair of (forward and reverse) primers suitable for amplifying a target locus; c) hybridizing the amplified DNA fragments obtained in step b) with a probe or primer that hybridizes with the target locus; d) detecting the hybrids formed in step c), and e) identifying microorganisms in the sample from the hybridization signals obtained in step d).

An example of comparable methods used on Mycobacterium spp. is described in U.S. 2013/0338018, which is herein incorporated by reference in its entirety.

### Methods of Genetic Characterization Using Third-Generation Sequencing

As many soil microorganisms remain unidentified and otherwise uncharacterized and without a reference genome available, in some embodiments, it is preferable to conduct whole genome sequencing and de novo genome assembly. The application of third-generation sequencing technology (TGS) in genetics/genomics and the overall decrease in cost for whole genome sequence (WGS) provide useful opportunities for genomic characterization of a wide variety of soil microorganisms. NGS platforms (e.g., Illumina) have many advantages, as they are cost-effective and high-throughput in nature. However, NGS technologies have disadvantages in genome assembly and analysis, for example, small read lengths, which creates challenges for de novo assembly, and de novo gene assemblies lacking significant portions of genomes and missing key genes for one or more reasons, like fragmentation. Gaps in genome information can create significant problems for de novo assembly, resulting in inaccurate taxonomic classification.

Similarly, the 16S rRNA gene has been a longtime cornerstone of sequence-based bacterial analysis, particularly at the species and strain level of microorganisms. In some instances, NGS technology targeting variable regions with short-read sequence platforms cannot provide the degree of taxonomic resolution that may be desirable to make small, yet significant distinctions between species or strains. The availability of third-generation technologies has increased the frequency and normalcy of high-throughput sequencing of the full 16S (1500 bp) gene. Thus, in some instances, for example, when detailed taxonomic resolution is desired, TGS technology may be used to sequence the entire 16S gene. Any suitable TGS platform may be used, for example, PacBio and Oxford Nanopore sequencing platforms, which are capable of producing reads in excess of 1500 bp (as compared to the 300 or less bp of Illumina). Example methods for full 16S rRNA gene sequencing and analysis are described in Johnson et al., Evaluation of 16S rRNA gene sequencing for specie and strain-level microbiome analysis, Nat. Com. 2019, 19:5029, which is herein incorporated by reference in its entirety.

TGS may also be utilized when de novo genome assembly and analysis are required or desired. The major platforms using TGS technology are Pacific Biosciences (PacBio) single-molecule real-time (SMRT) sequencing Oxford Nanopore Technologies (ONT) sequencing and BioNano Genomics (BioNano) sequencing. Additionally, several tools are being used in assembling long-read sequence technologies developed through TGS platforms based on de novo sequencing analysis in three different platforms including ONT, SMRT, and the BioNano sequencing platform. MinHash Alignment Process (MHAP), PBJelly, Hierarchical Genome-Assembly Process (HGAP), FALCON, and HINGE utilize long reads from SMRT platforms. A more detailed discussion of TGS technologies and assembly and analysis tools is described in Wee et al., The bioinformatics tools for the genome assembly and analysis based on third-generation sequencing, Brief. in Funct. Gen., 2019 18 (1), which is herein incorporated by reference in its entirety.

### Methods of Sequestering or Converting Carbohydrates in Microorganism Cells and Plants

Methods for sequestering carbohydrates in microorganism cells or plants are provided, whereby the carbohydrate is converted into a microorganism cell or plant, the method comprising introducing a carbohydrate into an environment that comprises one or more microorganism cells in a growth media, wherein the carbohydrate is used as a carbon source by the one or more microorganism cells in the growth media for growth or biosynthesis; converting the carbohydrate into the organic molecule products within the environment via a carbon-fixing reaction.

The one or more microorganism cells are bacterial cells, particularly soil microbes. In a further embodiment, the bacterial cells produce amino acids or proteins, or other biomass when cultured in the presence of a carbohydrate. In a further embodiment, the one or more microorganisms, biomass generated by the one or more microorganisms, or the plants are used as a source of food or nutrition for one or more other organisms. In an embodiment, the one or more other organisms include a plant or an animal.

The carbohydrate converted into a microorganism or plant may include one or more carbohydrates, particularly L-carbohydrates. Suitable carbohydrates include, without limitation, a pentose, a hexose, a heptose, or a combination thereof. More particularly, the carbohydrate may include, without limitation, ribose, ribulose, allose, glucose, mannose, talose, fructose, psicose, sedoheptulose, mannoheptulose, or a combination thereof.

Detection of the conversion/transformation of L-carbohydrates may determine whether L-carbohydrates persist in their original form, whether they are transformed into other compounds, taken up into the microbial biomass, and/or stabilized on soil mineral particles. For example, Moazeni et al. evaluated the utilization of chiral compounds in earth organisms. Although soil-, sediment- and lake-borne microbial communities prefer D-enantiomers, many can consume L-enantiomers if given time to acclimate. The methods of converting an L-carbohydrate into one or more microorganisms comprise collecting an environmental sample, such as a soil sample or a sample from a plant (e.g., a sample of plant-associated microorganisms, a plant part, a plant cell, etc.), preparing an isolate (for example, by using the methods described herein), and contacting the isolate with an L-carbohydrate to form a treated isolate. The treated isolate may be allowed to incubate with continuous shaking. L-carbohydrate presence may be quantified by the use of the colorimetric method. Further discussion of methods and materials for converting a carbohydrate, specifically an L-carbohydrate into a microorganism is provided in Moazeni et al., Imperfect Asymmetry of Life: Earth Microbial Communities Prefer D-Lactate but Can Use L-Lactate Also, Astrobiology, 2010, 10(4), which is herein incorporated by reference in its entirety.

These methods of incorporating an L-carbohydrate into a microorganism or plant may further be used to identify potential reactants that may be required for L-carbohydrate synthesis and/or precursor compounds of L-carbohydrates. Any suitable reactant may be used to synthesize L-carbohydrate compounds or derivatives thereof.

### Methods of Genome Modification of a Target Sequence in the Genome of a Plant or Plant Cell

The disclosure also relates to compositions and methods for genome modification of a plant target sequence in the genome of a plant or plant cell. The methods and compositions employ a guide RNA/Cas endonuclease system to provide an effective system for modifying or altering plant target sites within the genome of a plant, plant cell, or seed. Also provided are compositions and methods employing a guide polynucleotide/Cas endonuclease system for genome modification of a nucleotide sequence in the genome of a cell or organism, for gene editing, and/or for inserting or deleting a polynucleotide of interest into or from the genome of a cell or organism. Once a plant genomic target site is identified, a variety of methods can be employed to further modify the target sites such that they contain a variety of polynucleotides of interest. Breeding methods and methods for selecting plants utilizing a two-component RNA guide and Cas endonuclease system are also disclosed. Compositions and methods are also provided for editing a nucleotide sequence in the genome of a cell.

Any methods known in the art for modifying DNA in the genome of a plant can be used to modify genomic nucleotide sequences in planta, for example, to create or insert a gene or even to replace or modify an endogenous gene (or allele thereof) which impacts the synthesis or consumption of an L-carbohydrate. Such methods include, but are not limited to, genome-editing (or gene-editing) techniques, such as, for example, methods involving targeted mutagenesis, homologous recombination, and mutation breeding. Targeted mutagenesis or similar techniques are disclosed in U.S. Pat. Nos. 5,565,350; 5,731,181; 5,756,325; 5,760,012; 5,795,972, 5,871,984, and 8,106,259; all of which are herein incorporated in their entirety by reference. Methods for gene modification or gene replacement comprising homologous recombination can involve inducing double breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered endonucleases to make double-strand breaks at specific recognition sequences in the genome of a plant, other organism, or host cell. See, for example, Durai et al., (2005) Nucleic Acids Res 33:5978-90; Mani et al. (2005) Biochem Biophys Res Comm 335:447-57; U.S. Pat. Nos. 7,163,824, 7,001,768, and 6,453,242; Amould et al. (2006) J Mol Biol 355:443-58; Ashworth et al., (2006) Nature 441:656-9; Doyon et al. (2006) J Am Chem Soc 128:2477-84; Rosen et al., (2006) Nucleic Acids Res 34:4791-800; and Smith et al., (2006) Nucleic Acids Res 34:e149; U.S. Pat. App. Pub. No. 2009/0133152; and U.S. Pat. App. Pub. No. 2007/0117128; all of which are herein incorporated in their entirety by reference.

Unless stated otherwise or apparent from the context of use, the term “gene replacement” is intended to mean the replacement of any portion of a first polynucleotide molecule (e.g. a chromosome) that involves homologous recombination with a second polynucleotide molecule using a genome-editing technique as disclosed herein, whereby at least a part of the nucleotide sequence of the first polynucleotide molecule is replaced with the nucleotide sequence of the second polynucleotide molecule. It is recognized that such gene replacement can result in additions, deletions, and/or modifications in the nucleotide sequence of the first polynucleotide molecule and can involve the replacement of an entire gene or genes, the replacement of any part or parts of one gene, or the replacement of non-gene sequences in the first polynucleotide molecule.

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas. 1013133107; Scholze & Boch (2010) Virulence 1:428-432; Christian et al. Genetics (2010) 186:757-761; Li el al. (2010) Nuc. Acids Res. (2010) doi:10.1093/nar/gkq704; and Miller et al. (2011) Nature Biotechnology 29:143-148; all of which are herein incorporated by reference.

The CRISPR/Cas nuclease system can also be used to make double-strand breaks at specific recognition sequences in the genome of a plant for gene modification or gene replacement through homologous recombination. The CRISPR/Cas nuclease is an RNA-guided DNA endonuclease system comprising a complex of a Cas endonuclease and a guide polynucleotide that performs sequence-specific double-stranded breaks in a DNA segment homologous to the designed RNA. The Cas endonuclease unwinds the DNA duplex in close proximity to the genomic target site and cleaves both DNA strands upon recognition of a target sequence by a guide RNA, but only if the correct protospacer-adjacent motif (PAM) is approximately oriented at the 3′ end of the target sequence. In a guide RNA/Cas endonuclease system, the Cas endonuclease is guided by the guide RNA to recognize and optionally introduce a double-strand break at a specific plant target site into the genome of a cell. CRISPR loci (Clustered Regularly Interspaced Short Palindromic Repeats) (also known as SPIDRs—SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 bp depending on the CRISPR locus (WO2007/025097 published Mar. 1, 2007). It is possible to design the specificity of the sequence. See Cho S. W. et al., Nat. Biotechnol. 31:230-232, 2013; Cong L. et al., Science 339:819-823, 2013; Mali P. et al., Science 339:823-826, 2013; Feng Z. et al., Cell Research: 1-4, 2013, all of which are incorporated by reference in their entirety. CRISPR loci differ from other SSRs by the structure of the repeats, which have been termed short regularly spaced repeats (SRSRs). The repeats are short elements that occur in clusters, that are always regularly spaced by variable sequences of constant length.

In addition, a ZFN can be used to make double-strand breaks at specific recognition sequences in the genome of a plant for gene modification or gene replacement through homologous recombination. The Zinc Finger Nuclease (ZFN) is a fusion protein comprising the part of the FokI restriction endonuclease protein responsible for DNA cleavage and a zinc finger protein that recognizes specific, designed genomic sequences and cleaves the double-stranded DNA at those sequences, thereby producing free DNA ends (Urnov, et al., Nat Rev Genet. 11:636-46, 2010; Carroll D., Genetics. 188:773-82, 2011).

Breaking DNA using site-specific nucleases, such as, for example, those described herein, can increase the rate of homologous recombination in the region of the breakage. Thus, coupling of such effectors as described above with nucleases enables the generation of targeted changes in genomes which include additions, deletions, and other modifications.

The methods of the disclosure involve introducing a polynucleotide or polynucleotide construct into a plant. Methods for introducing polynucleotides or polynucleotide constructs into plants are known in the art including, but not limited to, stable transformation methods, transient transformation methods, and virus-mediated methods.

Depending on the desired outcome, the polynucleotides of the disclosure can be stably incorporated into the genome of the plant cell or not stably incorporated into the genome of the plant cell. If, for example, the desired outcome is to produce a stably transformed plant capable of synthesizing an L-carbohydrate and/or incapable of consuming an L-carbohydrate, then the relevant polynucleotide can be, for example, fused into a plant transformation vector suitable for the stable incorporation of the polynucleotide into the genome of the plant cell. As used herein, “incapable of consuming an L-carbohydrate” includes the complete inability to consume one or more L-carbohydrates and also the consumption of one or more carbohydrates at a rate slower than consumption of the L-carbohydrate's corresponding D-enantiomer. Typically, the stably transformed plant cell will be regenerated into a transformed plant that comprises in its genome the polynucleotide. Such a stably transformed plant is capable of transmitting the polynucleotide to progeny plants in subsequent generations via sexual and/or asexual reproduction. Plant transformation vectors, methods for stably transforming plants with an introduced polynucleotide, and methods for plant regeneration from transformed plant cells and tissues are generally known in the art for both monocotyledonous and dicotyledonous plants or described elsewhere herein.

In other embodiments in which it is not desired to stably incorporate the polynucleotide in the genome of the plant, transient transformation methods can be utilized to introduce the polynucleotide into one or more plant cells of a plant. Such transient transformation methods include, for example, viral-based methods which involve the use of viral particles or at least viral nucleic acids. Generally, such viral-based methods involve constructing a modified viral nucleic acid comprising a heterologous polynucleotide of the disclosure operably linked to the viral nucleic acid and then contacting the plant either with a modified virus comprising the modified viral nucleic acid or with the viral nucleic acid or with the modified viral nucleic acid itself. The modified virus and/or modified viral nucleic acids can be applied to the plant or part thereof, for example, in accordance with conventional methods used in agriculture, for example, by spraying, irrigation, dusting, or the like. The modified virus and/or modified viral nucleic acids can be applied in the form of directly sprayable solutions, powders, suspensions or dispersions, emulsions, oil dispersions, pastes, dustable products, materials for spreading, or granules, by spraying, atomizing, dusting, spreading or pouring.

Numerous plant transformation vectors and methods for transforming plants are available. See, for example, An, G. et al. (1986) Plant Physiol., 81:301-305; Fry, J., et al. (1987) Plant Cell Rep. 6:321-325; Block. M. (1988) Theor. Appl Genet. 76:767-774; Hinchee, et al. (1990) Stadler. Genet. Symp. 203212.203-212; Cousins, et al. (1991) Aust. J. Plant Physiol. 18:481-494; Chee, P. P. and Slightom, J. L. (1992) Gene. 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) Proc. Nat. Acad Sci. USA 90:11212-11216; Christou, P. (1993) In Vitro Cell. Dev. Biol.-Plant; 29P:119-124; Davies, et al. (1993) Plant Cell Rep. 12:180-183; Dong, J. A. and Mchughen, A. (1993) Plant Sci. 91:139-148; Franklin. C. I. and Trieu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi-Tech. 5: 17-27; Eapen et al. (1994) Plant Cell Rep. 13:582-586; Hartman, et al. (1994) Bio-Technology 12: 919923; Ritala, et al. (1994) Plant. Mol. Biol. 24:317-325; and Wan, Y. C. and Lemaux, P. G. (1994) Plant Physiol. 104:3748.

For the transformation of plants and plant cells, the nucleotide sequences of the disclosure can be inserted using standard techniques into any vector known in the art that is suitable for the expression of the nucleotide sequences in a plant or plant cell. The selection of the vector depends on the transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100: 247-250; Scheid et al., (1991) Mol. Gen. Genet., 228: 104-112; Guerche et al., (1987) Plant Science 52: 111-116; Neuhause et al., (1987) Theor. Appl Genet. 75: 30-36; Klein et al., (1987) Nature 327: 70-73; Howell et al., (1980) Science 208:1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, the stability of vectors used, the expression level of gene products, and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) Proc. Natl. Acad. Sci. USA 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the disclosure may be introduced into plants by contacting plants with a virus or viral nucleic acids. Generally, such methods involve incorporating a polynucleotide or polynucleotide construct of the disclosure within a viral DNA or RNA molecule. Further, it is recognized that promoters of the disclosure also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

If desired, the modified viruses or modified viral nucleic acids can be prepared in formulations. Such formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48. Perry's Chemical Engineer's Handbook, 4th Ed., McGraw-Hill, New York, 1963, pages 8-57 and et seq. WO 91/13546, U.S. Pat. Nos. 4,172,714, 4,144,050, 3,920,442, 5,180,587, 5,232,701, 5,208,030, GB 2,095,558, U.S. Pat. No. 3,299,566, Klingman, Weed Control as a Science, John Wiley and Sons, Inc., New York, 1961, Hance et al. Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants, and dispersants, preservatives, antifoaming agents, anti-freezing agents, for seed treatment formulation also optionally colorants and/or binders and/or gelling agents.

In certain embodiments, the polynucleotides of the disclosure can be provided to a plant using a variety of transient transformation methods known in the art. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) PNAS Sci. 91: 2176-2180 and Hush et al. (1994) J. Cell Science 107:775-784, all of which are herein incorporated by reference. Alternatively, the polynucleotide can be transiently transformed into the plant using techniques known in the art. Such techniques include the viral vector system and Agrobacterium tumefaciens-mediated transient expression as described elsewhere herein.

The cells that have been transformed may be grown into plants in accordance with conventional ways. See, for example. McCormick et al. (1986) Plant Cell Reports 5:81-84. These plants may then be grown, and either pollinated with the same transformed strain or different strains, and the resulting hybrid has constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide of the disclosure stably incorporated into their genome.

Unless expressly stated or apparent from the context of usage, the methods and compositions of the present disclosure can be used with any plant species including, for example, monocotyledonous plants and dicotyledonous plants. Examples of plant species of interest include, but are not limited to, corn (Zea mays), Brassica sp. (e.g., B. napus, B. rapa, B. juncea), alfalfa (Medicago sativa), rice (Oryza sativa), rye (Secale cereale), triticale (xTriticosecale or TriticumxSecale) sorghum (Sorghum bicolor, Sorghum vulgare), teff (Eragrostis tef), millet (e.g. pearl millet (Pennisetum glaucum), proso millet (Panicum miliaceum), foxtail millet (Setaria italica), finger millet (Eleusine coracana), switchgrass (Panicum virgatum), sunflower (Helianthus annuus), safflower (Carthamus tinctorius), wheat (Triticum aestivum), soybean (Glycine max), tobacco (Nicotiana tabacum), potato (Solanum tuberosum), peanut (Arachis hypogaea), cotton (Gossypium barbadense, Gossvpium hirsutum), strawberry (e.g. Fragariaxananassa, Fragaria vesca, Fragaria moschata, Fragaria virginiana, Fragaria chiloensis), sweet potato (Ipomoea batatus), yam (Dioscorea spp., D. rotundata, D. cayenensis, D. alata, D. polystachwya, D. bulbifera, D. esculenta, D. dumetorum, D. trifida), cassava (Manihot esculenta), coffee (Coffea spp.), coconut (Cocos nucifera), okra (Abelmoschus esculentus), oil palm (e.g. Elaeis guineensis, Elaeis oleifera), pineapple (Ananas comosus), citrus trees (Citrus spp.), cocoa (Theobroma cacao), tea (Camellia sinensis), banana (Musa spp.), avocado (Persea americana), fig (Ficus casica), grape (Vitis vinifera), guava (Psidium guajava), mango (Mangifera indica), olive (Olea europaea), papaya (Carica papaya), cashew (Anacardium occidentale), macadamia (Macadamia integrifolia), almond (Prunus amygdalus), date (Phoenix dactylifera), cultivated forms of Beta vulgaris (sugar beets, garden beets, chard or spinach beet, mangelwurzel or fodder beet), sugarcane (Saccharum spp.), oat (Avena sativa), barley (Hordeum vulgare), cannabis (Cannabis sativa, C. indica, C. ruderalis), poplar (Populus spp.), eucalyptus (Eucalyptus spp.), Arabidopsis thaliana, Arabidopsis rhizogenes, Nicotiana benthamiana, Brachypodium distachyon, tomato (Solanum lycopersicum), eggplant (Solanum melongena), pepper (Capsicum annuum), lettuce (e.g. Lactuca sativa), bean (Phaseolus vulgaris), lima bean (Phaseolus limensis), pea (Lathyrus spp.), chickpea (Cicer arietinum), and members of the genus Cucumis such as cucumber (C. sativus), cantaloupe (C. cantalupensis), and musk melon (C. melo), and ornamentals. Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. In certain embodiments, the plant is a tomato, cotton, bean, soybean, maize, beet, pepper, grape, sweet potato, cucurbit, or okra plant. According to some embodiments, the plant, plant part, or plant cell is all or a part of a tree, shrub, herb, grass, fern, moss, or agricultural crop. In a further embodiment, the agricultural crop is corn, rice, wheat, soybeans, sorghum, wheat bran, or other crop.

The present disclosure provides transformed plants, seeds, and plant cells produced by the methods of the present disclosure and/or comprising a polynucleotide impacting L-carbohydrate synthesis and/or L-carbohydrate consumption. Also provided are progeny plants and seeds thereof comprising a polynucleotide of the present disclosure. The present disclosure also provides fruits, seeds, tubers, leaves, stems, roots, and other plant parts produced by the transformed plants and/or progeny plants of the disclosure as well as biological samples comprising, or produced or derived from, the plants or any part or parts thereof including, but not limited to, fruits, tubers, leaves, stems, roots, and seed. In certain embodiments, the biological sample is a commodity plant product. In other embodiments, the transgenic plant is not a commodity plant product and is for example a tree, shrub, herb, grass, fern, moss, or other plant whose primary function is to sequester carbon.

The present disclosure additionally provides methods for identifying and/or selecting a plant capable of synthesizing an L-carbohydrate or incapable of consuming an L-carbohydrate. The methods comprise detecting in a plant, or in at least one part or cell thereof, the presence of a polynucleotide encoding a polypeptide involved in the ability to synthesize an L-carbohydrate or the inability to consume an L-carbohydrate. In certain embodiments, detecting the presence of the polynucleotide comprises detecting the entire nucleotide sequence in genomic DNA isolated from a plant. In certain embodiments, however, detecting the presence of the polynucleotide comprises detecting the presence of at least one marker within the nucleotide sequence, optionally wherein the marker encodes an amino acid mutation in or near the active center of the polypeptide.

In the methods for identifying and/or selecting a plant capable of synthesizing an L-carbohydrate or incapable of consuming an L-carbohydrate, detecting the presence of a polynucleotide the ability to synthesize an L-carbohydrate or the inability to consume an L-carbohydrate in the plant can involve one or more of the following molecular biology techniques that are disclosed elsewhere herein or otherwise known in the art including, but not limited to, isolating genomic DNA and/or RNA from the plant, amplifying a nucleic acid molecule comprising the polynucleotide and/or marker therein by PCR amplification, sequencing a nucleic acid molecule comprising the polynucleotide and/or marker, identifying the polynucleotide, the marker, or a transcript of the polynucleotide by nucleic acid hybridization, and conducting an immunological assay for the detection of the corresponding polypeptide encoded by the polynucleotide. It is recognized that oligonucleotide probes and PCR primers can be designed to identity the polynucleotides of the present disclosure and that such probes and PCR primers can be utilized in methods disclosed elsewhere herein or otherwise known in the art to rapidly identify one or more plants comprising the presence of a polynucleotide of the present disclosure in a population of plants.

Additionally provided are methods for introducing the ability to synthesize an L-carbohydrate or the inability to consume an L-carbohydrate. The methods comprise crossing (i.e., cross-pollinating) a first plant comprising in its genome a polynucleotide coding for a polypeptide involved in the ability to synthesize an L-carbohydrate or the inability to consume an L-carbohydrate with a second plant lacking in its genome such a polynucleotide. Either the first plant or the second plant can be the pollen donor plant. For example, if the first plant is the pollen donor plant, then the second plant is the pollen-recipient plant. Likewise, if the second plant is the pollen donor plant, then the first plant is the pollen-recipient plant. Following the crossing, the pollen-recipient plant is grown under conditions favorable for the growth and development of the plant and for a sufficient period of time for the seed to mature or to achieve an otherwise desirable growth stage for use. The seed can then be harvested and those seeds comprising the polynucleotide identified by any method known in the art.

### Orthologs

Also included in the disclosure are sequences that are orthologs of the disclosed sequences. As used herein, the terms “orthologous” or “ortholog” are used to describe genes or proteins encoded by those genes that are from different species but which have the same function (e.g., encode enzymes that catalyze the same reactions). Orthologous genes will typically encode proteins with some degree of sequence identity (e.g., at least 40%, 50%, 60%, 70%, 80%, 90%, or 95% sequence identity, conservation of sequence motifs, and/or conservation of structural features). Functions of orthologs are often highly conserved among species.

In certain embodiment, the orthologs contemplated herein have a nucleotide sequence having at least 80%, 85%, 90%, 91%, 92%, 93%, 94%, 95%, 96%, 97%, 98%, 99%, or greater nucleotide sequence identity to at least one target nucleotide sequence identified by the methods of genetic characterization described herein, and/or encode a polypeptide having least 80%, 85%, 90%, 91%, 92%, 93%, 94%, 95%, 96%, 97%, 98%, 99%, or greater amino acid sequence identity to the amino acid sequence identified by the methods described herein.

### Characterization of Soil Microbe L-Carbohydrate Consumption

After genetic and taxonomic characterization, soil microorganisms may be further characterized by their ability or inability to decompose L-sugars. Any suitable method of characterizing soil microbial L-carbohydrate consumption may be used. In an embodiment, the extent of L-sugar consumption is measured by contacting one or more L-sugars with soil microbes and recording the cumulative carbon dioxide— C respiration produced.

For example, methods of characterizing soil microbe L-sugar consumption can occur by providing one or more environmental samples; adding one or more L-sugars to the environmental samples; and tracking carbon dioxide emissions to determine whether L-sugar addition prompts increased microbial activity.

Methods of characterizing soil microbe L-sugar consumption can also occur through the preparation of assays, wherein growth media may be inoculated with a small sample of a specific microbial community from any given soil and extracted in buffered saline extraction by vortexing. Assays may be used to detect consumption (CO₂ production) of the single L-carbohydrate carbon source included in the growth media. Upon detection of CO₂ production, samples may be diluted by orders of magnitude across dozens of new replicate samples in order to identify individual taxa or consortia capable of L-carbohydrate consumption.

### Characterization of Soil Microbe L-Carbohydrate Synthesis

The initial communities of microorganisms, particularly those grown through the use of general growth media and identified via isolation or other methods, may comprise a subset of microbes not capable of consuming L-carbohydrates. The methods described herein may include a further step of tracking the concentrations of L-carbohydrates in environmental samples or isolated microorganisms on growth media from an initial point and over a period of time following exposure to an L-sugar or other carbohydrate. The detection of increasing concentrations of unconsumed L-carbohydrates in the samples indicates the presence of microorganisms capable of L-carbohydrate anabolism. Upon detection of increased concentrations of unconsumed L-carbohydrates, samples comprising the unconsumed L-carbohydrates may be diluted by orders of magnitude across multiple new replicate samples in order to identify individual taxa or consortia capable of L-carbohydrate synthesis.

Additional and/or alternative methods of identifying and characterizing soil microbes that synthesize L-carbohydrates include, for example, high-throughput assays capable of detecting L-sugar synthesizers that also utilize liquid chromatography-mass spectrometry technology. A benefit of using high-throughput assays is the volume of samples that can be characterized. For example, crowd-sourcing a large number of sample soils and analyzing the same through high-throughput screening

### Methods of Identifying and Characterizing D- and L-Carbohydrate Catabolic and Anabolic Pathways and Substrates

Methods of characterizing carbohydrate metabolism in organisms, particularly L-carbohydrate metabolism in microorganisms are provided. Relatedly, methods of identifying enzymes, enzyme substrates, enzyme kinetics, and efficiency are also provided. Bacterial carbohydrate metabolism is extremely diverse both due to the volume of soil microorganisms and because carbohydrates are involved in a variety of cellular processes. Often, however, bacterial genes belonging to the same metabolic pathway are often co-localized in the chromosome. Co-localized genes form cassettes, ranging in size from two to fifteen genes.

Two major factors influencing the cassette-forming tendency are gene function and bacterial phylogeny. Significantly, genes from several different functional classes tend to co-localize with genes from the same class, indicating the important role of the clustering of genes with similar functions. This clustering is important when identifying the genes encoding carbohydrate transforming enzymes, such as hydrolases, phosphorylases, dehydratases, acetylases, and the related transporters and transcription regulators. A further description of methods of analyzing the co-localization of functions and genes involved in carbohydrate metabolism, along with a comparison of bacteria gene sequences is provided in Kaznadzey et al., Sugar Lego: gene composition of bacterial carbohydrate metabolism of genomic loci, Bio. Dir. 2017, 28, which is herein incorporated by reference in its entirety.

Any suitable method of identifying L-carbohydrate catabolic and anabolic pathways may be utilized, including methods of characterizing the occurrence of L-carbohydrates, their metabolism in plants, and their genetic regulation known in the art. The anabolism, catabolism, and regulation of L-carbohydrates are often closely entwined with that of D-carbohydrates. For example, with respect to L-galactose, plants can contain several Levo forms of carbohydrates like L-arabinose, L-fucose, L-rhamnose, and L-galactose. Both D- and L-galactose are synthesized from UDP-D-glucose (via UDP-galactose) when D-galactose is more abundant in plants. Separation of the enantiomeric forms showed the ratio of D-/L-galactose ranged from 7.3 to 70, in the lycophytes or spinach cells. Further description of carbohydrate enantiomers and their role in the ecosystem is provided in Lojkova et al., Enantiomers of Carbohydrates and Their Role in Ecosystem Interactions: A Review. Symmetry 2020, 12, 470, which is herein incorporated by reference in its entirety. Much research has been conducted identifying the L-galactose pathway, the linear form of which is a major biosynthetic route to L-ascorbate (vitamin C) production in higher plants. GDP-L-galactose phosphorylase functions in concert with other enzymes of the L-galactose pathway to provide plants with the appropriate levels of L-ascorbate. Further discussion of the L-galactose pathway is provided in Linster & Clarke, L-ascorbate biosynthesis in higher plants: the role VTC2, Trends in Plant Science 2008, 13(11), which is herein incorporated by reference in its entirety.

Methods of identifying and characterizing L-glucose catabolic and anabolic pathways have also been developed. For example, Shimizu et al. discuss the isolation of an L-glucose-utilizing bacterium, Paracoccus sp. 43P, from the soil by enrichment cultivation in a medium containing L-glucose as the carbon source. It was determined that NAD⁺-dependent L-glucose dehydrogenase has sole activity toward L-glucose. This enzyme, LgdA, and the lgdA gene was found to be located in a cluster of putative inositol catabolic genes. Shimizu et al. also discuss methods of enzyme purification and gene cloning which revealed that the corresponding gene resides in a nine-gene cluster, the lgn cluster. Further methods relating to the kinetic and reaction product analysis of each gene product in the cluster are also provided. Shimizu et al. further describe methods for gene disruption studies that are capable of confirming whether the identified genes are in fact responsible for L-glucose catabolism. Shimizu et al., An L-glucose Catabolic Pathway in Paracoccus Species 43P, J. Biol. Chem. 2012, 287(48), and all the methods described therein are herein incorporated by reference in its entirety. The experimental methods of Shimizu et al. may be generalized and applied to methods of ascertaining the catabolic pathway for any L-carbohydrate described herein for any one of the microbes collected from environmental samples described herein.

Similarly, Fukano et al. discuss L-glucose catabolic pathways in Paracoccus laeviglucosivorans and the relevant genes involved. Fukano et al.'s methods of catabolic pathway identification may be used according to the present disclosure. Fukano et al., Structural basis of L-glucose oxidation by scyllo-inositol dehydrogenase: Implications for a novel enzyme subfamily classification, PLoS ONE, 2018, 13(5) is herein incorporated by reference in its entirety.

More generally, plants produce a variety of compounds through metabolic pathways, both primary metabolism and secondary metabolism. The collection of biochemical processes and the compounds involved which are essential for the growth and survival of the plant are considered primary metabolic pathways and their products. Primary metabolism is generally considered to encompass those biochemical processes that lead to the formation of primary sugars, (such as glucose), amino acids, common fatty acids, nucleotides, and the polymers derived from them (polysaccharides such as starch, proteins, lipids, RNA and DNA, etc.). Secondary metabolism can be defined as those biochemical processes that are not essential to all cells, microbial or plant. For example, secondary metabolic pathways can determine such plant features as color, taste, morphology, etc. The manipulation of metabolic pathways, particularly primary metabolic pathways can produce novel compositions of biochemicals or produce plant tissue with altered content. In some embodiments, manipulation of primary metabolic pathways results in the presence of one or more L-carbohydrates, or the inability to decompose the same. In other embodiments, manipulation of secondary pathways impacts the synthesis or decomposition of L-carbohydrates.

In some embodiments, a primary metabolism of a plant or microorganism has been altered successfully to produce a novel phenotype that represents a compositional change or a reduction or elimination of a specific substance (e.g., D-carbohydrates). Typically, these manipulations have been accomplished by ectopic expression of a gene, for example, over-expressing a plant gene in certain tissues or a constitutive fashion rather than a regulated fashion, or by inhibition of specific gene activity by antisense RNA, ribozymes, or co-suppression.

The expression of an enzyme can be modified at many levels. This includes control at the gene expression level, translation, protein processing, and allosteric control of protein function. Thus ectopic expression of a gene involved in primary metabolism may not overcome the complex biochemical controls on the regulation of primary metabolism. Furthermore, redundancy in primary metabolism also poses a difficult hurdle to overcome in these manipulations since primary metabolic pathways are essential to plant growth and survival.

The present disclosure relates to methods to target the formation of a primary metabolite. In some embodiments the method comprises altering the availability of a substrate that is specific to a metabolic pathway and essential to the formation of the final metabolic product, particularly those compounds within one to ten biochemical steps of final product formation. Targeting substrates at steps near the final product formation avoids the problems associated with alterations of other, non-targeted metabolites involved in primary pathways since it occurs after the entry point of a substrate into the primary metabolic pathway. Thus the method provides a novel means of specifically targeting the reduction or alteration of secondary metabolites by identifying precursors used within a secondary metabolic pathway that do not comprise substrates for primary metabolic pathways.

The method can also comprise altering the availability of a substrate in a tissue-specific manner such that only certain tissues, e.g. seed tissues, are altered. Thus the methods disclosed herein provide a means of specifically targeting the reduction or alteration of metabolites by identifying precursors used within a metabolic pathway.

As described herein, the present disclosure relates to methods of making transgenic plants. In the context of metabolic pathways, the methods comprise a) introducing into a plant cell capable of being transformed and regenerated to a whole plant a DNA expression cassette comprising, in addition to DNA sequences required for transformation and selection in plant cells, a DNA sequence that, under the control of a promoter active in plant cells, encodes a protein capable of modifying the utilization of a substrate in a metabolic pathway, wherein the substrate is preferably metabolite of a carbohydrate, and b) recovering a plant which has an altered content of at least one product of the metabolic pathway. In another embodiment, the disclosure provides a method for making a genetically transformed seed comprising growing the plant obtained according to steps a) and b) of the method described above under conditions that permit the formation of seed.

The method is not restricted to any particular secondary metabolic pathway. Nor is the method restricted to any particular plant species. Rather, the method can be applied to alter metabolic pathways common to many commercially valuable crop species, including monocots and dicots, or to target a metabolite relevant for the synthesis or consumption of L-carbohydrates.

The biochemical basis for the methods is founded on the concept of the regulation of enzyme activity by the availability of substrates. In general, enzymatic rates are influenced by the availability of substrates. In other words, an enzyme will produce a product at a rate proportional to the amount of available substrate. The reduction of substrate concentration leads to lower levels of the product. Furthermore, many enzymes are subject to end-product inhibition, meaning the enzymatic rate is reduced in the presence of a large excess of the final product. Hence, by altering the levels of available substrates or enzymatic products, the overall production of compounds produced by a biochemical pathway can be changed.

Discussion of methods for modifying metabolic compounds in plants is discussed in U.S. Pat. No. 6,703,539, which is herein incorporated by reference in its entirety.

### EXAMPLES

### Example 1. Soil Sample Collection and L-Carbohydrate Consumption

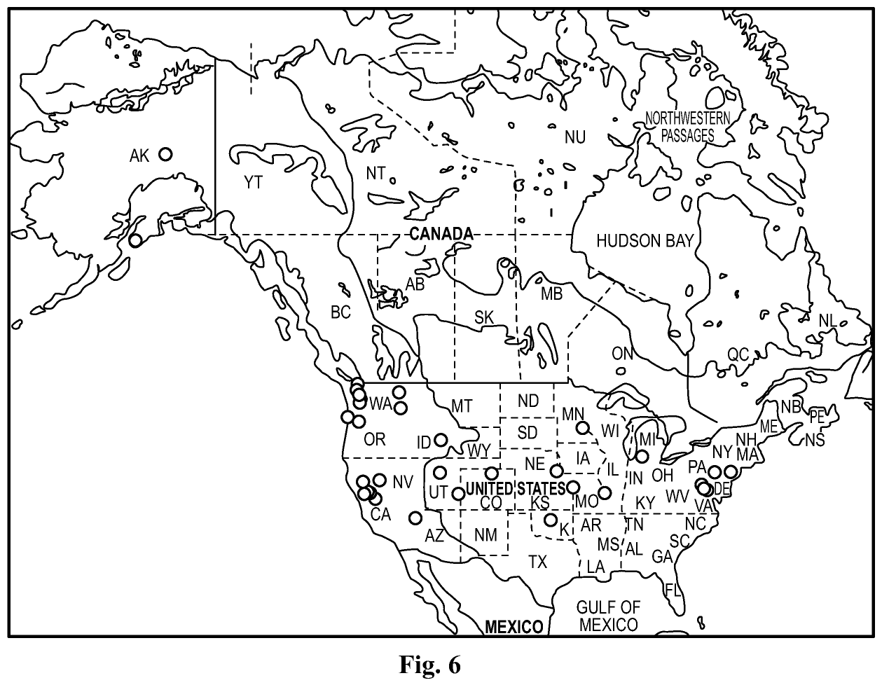

Soil samples were collected at a range of locations across the United States, as shown in FIG. 6. Samples originated from the four soil orders predominant in US agricultural soils (Mollisols, Alfisols, Ultisols, and Entisols) and spanned aridic to typic moisture conditions and mesic to thermic temperature regimes. Soil texture ranged from coarse to very fine.

In particular, soil samples were crowd-sources, building a collection of 62 soils representing several soil types from sites across the country and from states such as Alaska, Washington, Oregon, California, Nevada, Utah, Idaho, Colorado, Oklahoma, Nebraska, Minnesota, Missouri, Michigan, Pennsylvania, Connecticut, Delaware, West Virginia, and Virginia.

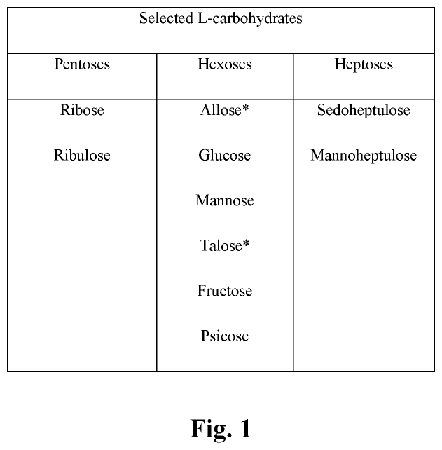

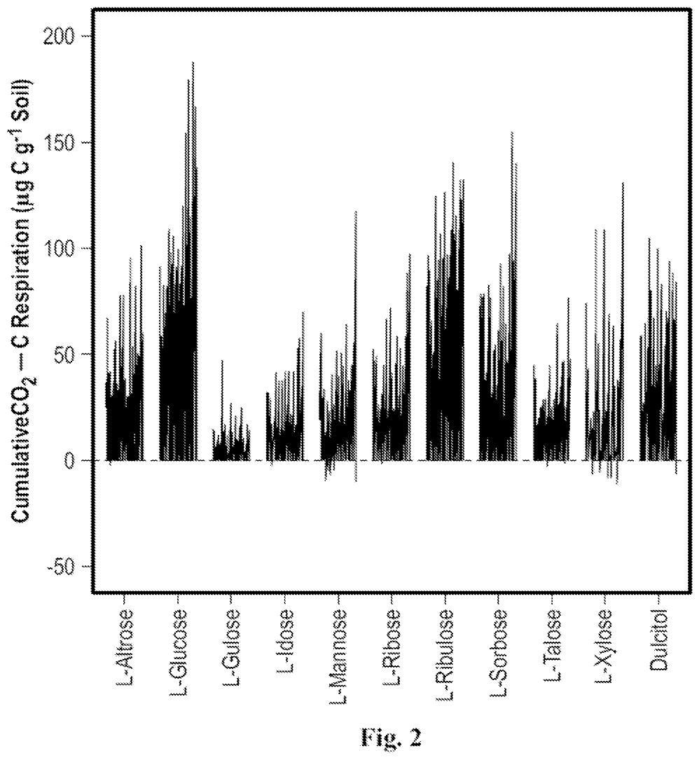

Microbes in the soil samples were then evaluated for their capability to consume L-carbohydrates. Respiration was induced following sugar additions for ten L-carbohydrates (or L-sugars) as identified in FIG. 1 and the rare, non-chiral sugar dulcitol. The cumulative CO₂—C respiration was recorded using methods similar to those described in the USDA article “Soil Respiration.” (nrcs.usda.gov/Internet/FSE_DOCUMENTS/nrcs142p2_051573.pdf) which s herein incorporated by reference in its entirety. The results are shown in FIG. 2.

As demonstrated by FIG. 2, microbes in all soils tested were capable of consuming several L-carbohydrates, including the non-chiral sugar dulcitol (a rare sugar also known as galactitol). Each bar represents the average L-sugar-induced respiration for one of 62 soils examined. These sugars were consumed when added to soils, indicating that these L-sugars alone may be poor candidates for carbon storage.

### Example 2. CO₂—C Respiration of Microbes in Contact with Four Additional L-Carbohydrates

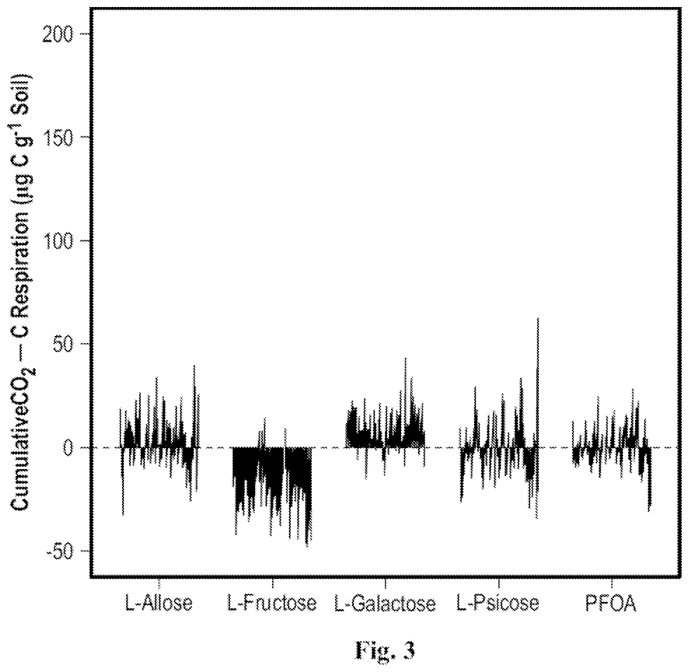

The cumulative CO₂—C respiration achieved by soil microbes for four additional L-carbohydrates, specifically decomposition-resistant L-allose, L-fructose, L-galactose, and L-psicose as referenced in FIG. 1, was ascertained. Specifically, microbes in several soil samples were evaluated for their capability to consume L-carbohydrates. Soil samples were collected, the samples comprising one or more microorganisms. Respiration was induced following sugar additions for the four L-carbohydrates The cumulative CO₂—C respiration was recorded and is shown in FIG. 3.

As shown in FIG. 3, the results indicate that the four L-carbohydrates were not consumed by microbes in any of the soils tested. The very low respiration induced by these four L-sugars was similar to that induced by perfluorooctanoic acid (PFOA), also known as forever plastic. The results further indicate the addition of L-fructose inhibited consumption of soil organic matter.

Thus, in contrast to the results of Example 1, Example 2 produced results indicating that four L-sugars (L-allose, L-fructose, L-galactose, and L-psicose) were only marginally consumed by soil microbes in several soil types. Respiration rates comparable to PFOAs indicate substantial decomposition resistance for the four L-sugars. Identification of these rare L-sugars that persist in soil beneficially creates substantial opportunities for carbon sequestration. When carbon is fixed in the form of L-allose, L-fructose, L-galactose, and/or L-psicose, that carbon will not be released as atmospheric carbon dioxide but will instead persist in L-carbohydrate form because a substantial number of soil types contain microbes that do not consume these four L-sugars.

### Example 3. Microbial Diversity

In order to examine the taxonomic diversity captured in the soil source collection, 16S rRNA V4 amplicon libraries of DNA preparations were sequenced using standard methods as described in Caporaso, J. Greg, Gail Ackermann, Amy Apprill, Markus Bauer, Donna Berg-Lyons, Jason Betley, Noah Fierer, et al. “EMP 16S Illumina Amplicon Protocol.” protocols.io, (Apr. 14, 2018), which is herein incorporated by reference in its entirety.

The sequences were analyzed for classification and abundance using protocols described in Bolyen, Evan, Jai Ram Rideout, Matthew R. Dillon, Nicholas A. Bokulich, Christian C. Abnet, Gabriel A. Al-Ghalith, Harriet Alexander, et al. “Reproducible, Interactive, Scalable and Extensible Microbiome Data Science Using QIIME 2.” Nature Biotechnology 37, no. 8 (August 2019): 852-57 and Straub, Daniel, Nia Blackwell, Adrian Langarica-Fuentes, Alexander Peltzer, Sven Nahnsen, and Sara Kleindienst. “Interpretations of Environmental Microbial Community Studies Are Biased by the Selected 16S rRNA (Gene) Amplicon Sequencing Pipeline.” Frontiers in Microbiology 0 (2020), both of which are herein incorporated by reference in their entirety.

A taxonomic comparison of the 12,382 OTUs was done comparing the instant collection vs. a large global survey as described in Delgado-Baquerizo, Manuel, Angela M. Oliverio, Tess E. Brewer, Alberto Benavent-Gonzalez, David J. Eldridge, Richard D. Bardgett, Fernando T. Maestre, Brajesh K. Singh, and Noah Fierer. “A Global Atlas of the Dominant Bacteria Found in Soil.” Science 359, no. 6373 (Jan. 19, 2018): 320-25, which is herein incorporated by reference in its entirety.

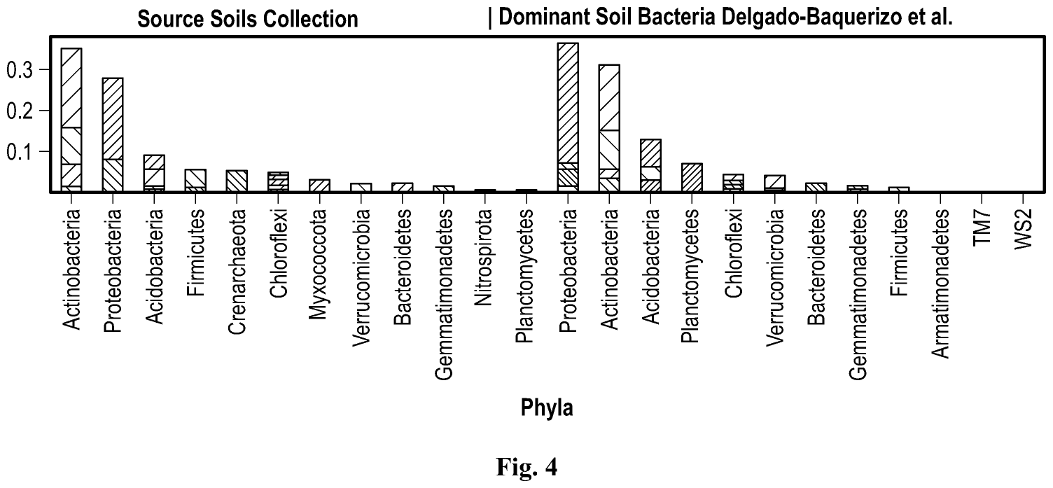

This comparison found that 511 bacteria taxa are dominant in abundance across almost all soils. The dominant bacteria taxa comparison is shown in FIG. 4, wherein identical colors/patterns are matching classes.

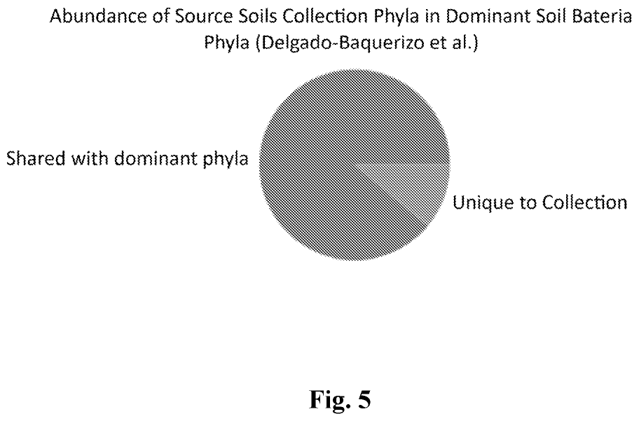

Relatedly, FIG. 5 shows the abundance-weighted percentage of shared phyla.

The embodiments being thus described, it will be obvious that the same may be varied in many ways. Such variations are not to be regarded as a departure from the spirit and scope of the disclosure and all such modifications are intended to be included within the scope of the following claims.

What is claimed is:

1. A method of sequestering carbon comprising:
providing a composition comprising one or more microorganisms incapable of decomposing at least one L-carbohydrate; and
contacting the composition with a soil source.

2. The method of claim 1, wherein the at least one L-carbohydrate comprises an L-pentose, L-hexose, L-heptose, or a combination thereof.

3. The method of claim 1, wherein the at least one L-carbohydrate comprises L-altrose, L-glucose, L-gulose, L-idose, L-mannose, L-ribulose, L-sorbose, L-talose, L-xylose, L-allose, L-fructose, L-galactose, L-psicose, or a combination thereof.

4. The method of claim 1, wherein the composition further comprises one or more microorganisms capable of synthesizing at least one L-carbohydrate.

5. The method of claim 1, wherein the one or more microorganisms comprise an Actinobacteria, Proteobacteria, Acidobacteria, Firmicutes, Crenarchaeota, Chloroflexi, Myxococcota, Verrucomicrobia, Bacteroidetes, Gemmatimonadetes, Nitrospirota, Planctomycetes, Proteobacteria, or a combination thereof.

6. The method of claim 1, further comprising isolating the one or more microorganisms from an environmental sample.

7. The method of claim 6, wherein the isolation of the one or more microorganisms comprises:
collecting an environmental sample;
serially diluting the sample to form a suspension comprising one or more microorganisms;
growing the one or more microorganisms on a growth media;
selecting one or more microorganisms incapable of decomposing at least one L-carbohydrate; and
separating the one or more microorganisms from the growth media.

8. A method of sequestering carbon in a transgenic plant comprising:
a) introducing into a plant, plant part, or plant cell a DNA expression cassette comprising, a DNA sequence that encodes a protein capable of modifying the plant, plant part, or plant cell's utilization of a substrate in a metabolic pathway; and
b) generating a plant cell that has at least one product of the metabolic pathway.

9. The method of claim 8, wherein the product of the metabolic pathway is an L-carbohydrate.

10. The method of claim 9, wherein the L-carbohydrate comprises an L-pentose, L-hexose, L-heptose, or a combination thereof.

11. The method of claim 10, wherein the L-carbohydrate comprises L-altrose, L-glucose, L-gulose, L-idose, L-mannose, L-ribulose, L-sorbose, L-talose, L-xylose, L-allose, L-fructose, L-galactose, L-psicose, or a combination thereof.

12. The method of claim 8, wherein the plant, plant part, or plant cell is all or a part of a tree, shrub, herb, grass, fern, moss, or agricultural crop.

13. The method of claim 12, wherein the agricultural crop is corn, rice, wheat, soybeans, sorghum, wheat bran, or other crop.

14. A method of converting an L-carbohydrate into one or more microorganisms comprising:
introducing one or more L-carbohydrates into an environment that comprises cells of the one or more microorganisms in a growth media;
wherein the one or more L-carbohydrates are used as a carbon source by the cells of the one or more microorganisms in the growth media for growth or biosynthesis; and
converting a part or all of the one or more L-carbohydrates into the one or more microorganisms within the environment via a carbon-fixing reaction.

15. The method of claim 14, wherein the one or more L-carbohydrates comprise an L-pentose, L-hexose, L-heptose, or a combination thereof.

16. The method of claim 15, wherein the one or more L-carbohydrates comprise L-altrose, L-glucose, L-gulose, L-idose, L-mannose, L-ribulose, L-sorbose, L-talose, L-xylose, L-allose, L-fructose, L-galactose, L-psicose, or a combination thereof.

17. The method of claim 14, wherein the one or more microorganisms comprises a bacterium, archaeon, fungus, alga, protozoan, virus, or a combination thereof.

18. The method of claim 16, wherein the bacterium comprises an Actinobacteria, Proteobacteria, Acidobacteria, Firmicutes, Crenarchaeota, Chloroflexi, Myxococcota, Verrucomicrobia, Bacteroidetes, Gemmatimonadetes, Nitrospirota, Planctomycetes, Proteobacteria, or a combination thereof.

19. A kit for detecting and identifying one or more microorganisms capable of synthesizing an L-carbohydrate and/or not decomposing an L-carbohydrate comprising:
a) a composition comprising at least one primer pair comprising a forward primer and a reverse primer, suitable for amplifying a locus involved in the synthesis of an L-carbohydrate and/or the inability to digest an L-carbohydrate;
b) a composition comprising at least one nucleic acid probe capable of hybridizing to the locus;
c) a buffer suitable for hybridizing a reaction between a nucleic acid target and the probe and/or primer pair, wherein the reaction forms one or more hybridized nucleic acids;
d) a solution for washing the one or more hybridized nucleic acids; and
e) optionally, a means for detection of the one or more hybridized nucleic acids.

20. A method for detecting and/or identifying one or more microorganisms capable of synthesizing an L-carbohydrate and/or not decomposing an L-carbohydrate comprising:
a) isolating a target DNA sequence involved in the synthesis of an L-carbohydrate and/or the inability to consume an L-carbohydrate;
b) amplifying the target DNA sequence with at least one primer pair comprising a forward primer and a reverse primer, suitable for amplifying a locus involved in the synthesis of an L-carbohydrate and/or the inability to digest an L-carbohydrate;
c) contacting the target DNA sequence or a fragment thereof with a probe and/or a primer that hybridizes with the target DNA sequence to form a hybrid;
d) detecting the hybrid; and
e) identifying one or more microorganisms capable of synthesizing an L-carbohydrate and/or not decomposing an L-carbohydrate.

21. A microorganism produced by the method of claim 14.

22. A plant, plant part, or plant cell produced by the method of claim 8.
